(19) United States
(12) Patent Application Publication
(10) Pub. No.: US 2015/0282976 A1
(43) Pub. Date: Oct. 8, 2015

(54) KIT AND METHOD FOR MAKING A SPLINT

(71) Applicant: MakeItMarketUSA.com, LLC., Charlotte, NC (US)

(72) Inventors: Katherine Liddle, Galloway, NJ (US); Michael Murdock, Charlotte, NC (US); William Medina, Highlands Ranch, CO (US)

(21) Appl. No.: 14/470,276

(22) Filed: Aug. 27, 2014

Related U.S. Application Data
(60) Provisional application No. 61/995,225, filed on Apr. 7, 2014.
(60) Provisional application No. 62/021,442, filed on Jul. 7, 2014.

(51) Int. Cl. A61F 5/058

(52) U.S. Cl. CPC A61F 5/05841; A61F 2210/0071; A61F 2210/0076

(57) ABSTRACT

A kit and method are presented for making a mending device, such as a splint, for immobilizing a body part, such as a limb. The kit includes a formable material and a heating source. The heating source is used to selectively heat the formable material to an elevated material, molding the formable material into a required shape for the mending device and allowing the molded material to cool to room temperature and become hard again. The formable material maybe molded on or around and in contact with the desired body part. The heating source may include a chemical composition that is selectively activated, for example by adding water to it.

### RELATED APPLICATIONS

This application claims priority to U.S. Provisional Patent Applications No. 61/995,225 filed Apr. 7, 2014, and 62/021,442 filed on Jul. 7, 2014, both incorporated herein by reference in their entirety.

### BACKGROUND OF THE INVENTION

A. Field of Invention

This invention pertains to a kit and method for mending or strengthening a patient's limb and, in particular, to a kit and method for making a device for mending a broken limb such as a finger, an arm, a leg of a person or animal, using a material that is hard and rigid at room temperature but because soft and malleable at higher temperatures. The kit includes the material and a heating source for heating the material.

B. Description of the Prior Art

There are many splinting or strengthening or repair systems available today. For examples, instructions for addressing the problem of mending a broken limb, especially in the field, or other environments where there is no immediate medical (or veterinary) assistance available, include finding sticks, twigs or other stiffening rod-shaped elements, positioning these elements adjacent to the broken limb (preferably on opposite sides thereof) and then tying them tightly together thereby immobilizing the limb. Then, the body heals itself by generating new tissues to fuse the broken ends of the bones together. If the process is done correctly, the limb will heal properly. If it is done incorrectly, the broken sections may grow together in an improper position, and surgical and/or other procedures must be used to reset the bone.

Systems are also presently available for use by professionals to mend broken limbs. There are kits and methods available which comprise materials and instructions for splint or strengthening or repair systems to be used in temporary or emergency situations to manage fractures or other injuries, including fractures or injuries to the jaw areas of humans and animals and to manage repairs for objects. Important factors for consideration in choosing a splinting material and method include: 1) a medical or other professional user may not be readily available, and 2) the patient or non-professional user may or may not be required to form the device on their own, and 3) forming the splint, strengthening or repair device by a patient or non-professional user on their own may require activities which the patient or non-professional user may or may not be able to achieve, and 4) the patient or user or object may be subject to further injury or damage if immediate splinting or strengthening or repair is not accomplished, and 5) the patient or a non-professional user or consumer may not be capable of following the kit and method instructions correctly and in a timely manner.

### SUMMARY OF THE INVENTION

The goal of this invention is to create a kit and method of using the same for making a mending device, such as a splint adapted to mend the limb of a person. The kit includes instructions which are intuitive and easy to understand for both professional and non-professional users alike. intuitive or easy to understand instructions allow the user to focus on creating a device of this invention well suited for its intended purpose.

In one embodiment, the present invention pertains to a kit including a formable material and a heating source. The heating source may be a chemically activated source that is selectively activated on demand to warm the formable material. The heating source may be for example a flameless heating compound or mixture of compounds that is activated on contact with air, water, mechanical manipulation so on.

The formable material is a material that has a hard, rigid shape at room temperature and above (up to a threshold temperature range). As the material is heated above the threshold temperature range, it becomes malleable. As the material is heated above this temperature, it becomes soft and malleable so that it can be molded into various shapes. The material maintains its molded shape after it cools down to room temperature.

After it is softened, the formable material is shaped to form a splint or similar reinforcing structure for the limb of a patient or animal. Optionally, wrapping tape may be added on top of the splint or between the splint and the patient's tissues.

Easy to read instructions are also included for using the kit to make a mending device.

### BRIEF DESCRIPTION OF THE DRAWINGS

For the purpose of illustrating the invention, there is shown in the accompanying drawings a splint for patient's limb being formed in accordance with the invention; it being understood that the invention is not intended to be limited to the precise arrangements, compounds and instrumentalities shown.

FIG. 1 shows the components of the kit for making a mending device such as a splint on accordance with this invention, together with some splints made using the kit;

FIG. 1A is a flow chart of the method used to make the splint of FIG. 1;

FIG. 2 shows pieces of the formable material being positioned for wrapping;

FIG. 3 shows the bag used as a heating source for heating the formable material;

FIG. 4 shows the bag with the formable material being heated;

FIG. 5 shows the wrapped formable material having been just removed from the flameless heater bag;

FIG. 6 shows the unwrapped formable material which is now ready to be formed as desired;

FIG. 7 shows the formable material being shaped;

FIG. 8 shows a splint 20 made on a finger form the formable material of FIG. 7; material being formed more completely and being used as a temporary or emergency splinting device for a fractured finger; and

FIG. 9 shows the underside of the finger of FIG. 8 that is not covered by material.

### DETAILED DESCRIPTION OF THE INVENTION OF THE INVENTION

This invention is intended to provide an easy to use kit and method for making a splint or other similar repair or strengthening device for a human or animal or object. A kit for making the above described device comprises a thermoplastic or other formable material of chemistry, color, size and shape and thickness to suit particular or individual applications. For example, different chemistries, colors, sizes, shapes and thicknesses may be required depending on intended use by multiple industries: military, hospital, veterinary, dental, surgical, sports, retail or consumer using the invention and as well on whether the invention is to be used within the U.S. or in other countries. The kit also includes instructions for making said device.

In one embodiment, the present invention pertains to a kit including a formable material and a heating source. The heating source may be a chemically activated source that is selectively activated to warm the formable material. The heating source may be for example a flameless heating compound or mixture of compounds that is activated on contact with air, water, mechanical manipulation so on.

The term “formable material” is used herein to define a material that has a solid, non-malleable shape at room temperature and above (up to a threshold temperature). The threshold temperature may be for example about 37°±5° C. As the material is heated above this temperature, it becomes soft and malleable so that it can be molded into various shapes. For example, the formable material may become sufficiently malleable so that it can be shaped as desired in the range of 45-75° C.

The term “mending device” is used to define a hard, rigid device at room temperature that can or has been applied to a portion of the body of the patient (both human and veterinary) to maintain that body portion stable and prevent its movement, for example, while a natural healing takes place. The mending device could be a splint and could be applied to the limbs of a person or animal or to other body portions.

The formable material may have several compositions. In one embodiment the compositions are biocompatible. For example, the formable material may be made of polycaprolactone, optionally with small amounts of additives such as a colorant. The formable material may also be from other compounds, including compounds made from a polyvinyl acetate or a thermoplastic material.

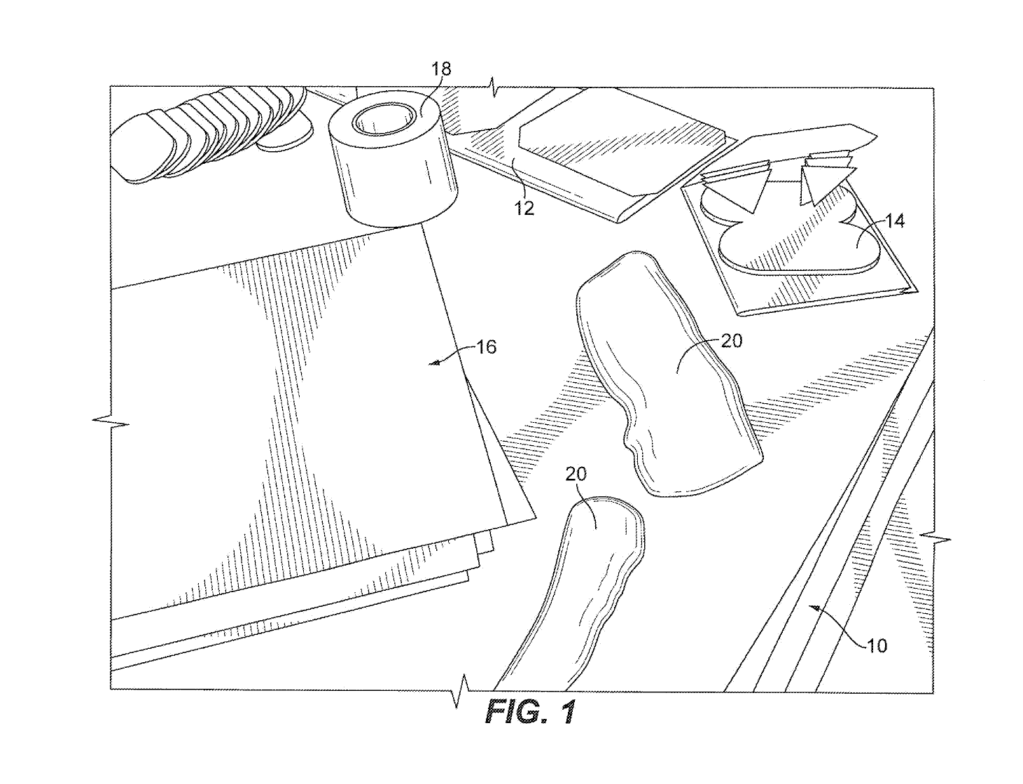

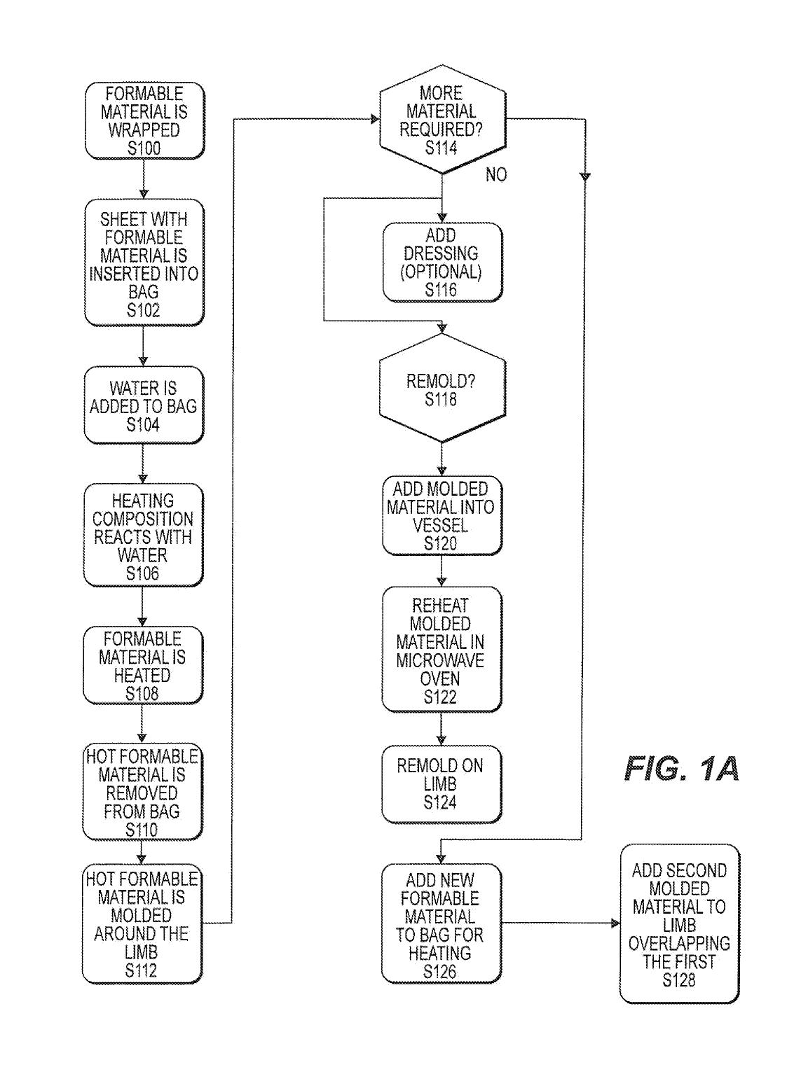

Referring to FIG. 1, a kit 10 constructed in accordance with this invention includes a bag 12 with the heating composition (not shown), one or more pieces of the formable material 14, one or more sheets of paper 16, and, optionally a roll of tape 18 or other means of dressing a limb of a patient.

The bag 12, as previously discussed, includes a heating composition, such as the ones described U.S. Pat. No. 5,517,981 to Taub. This composition heats when a water is added to it Other well known heating compositions may be used as well.

The formable material 14 is provided in wafers or sheets sized and shaped for various splints. For example, the material can be provided in 4 in×4 in square pieces, or in pieces that have been cut-out so that they have a somewhat elliptical shape as shown. Each material 14 may be a couple of millimeters thick.

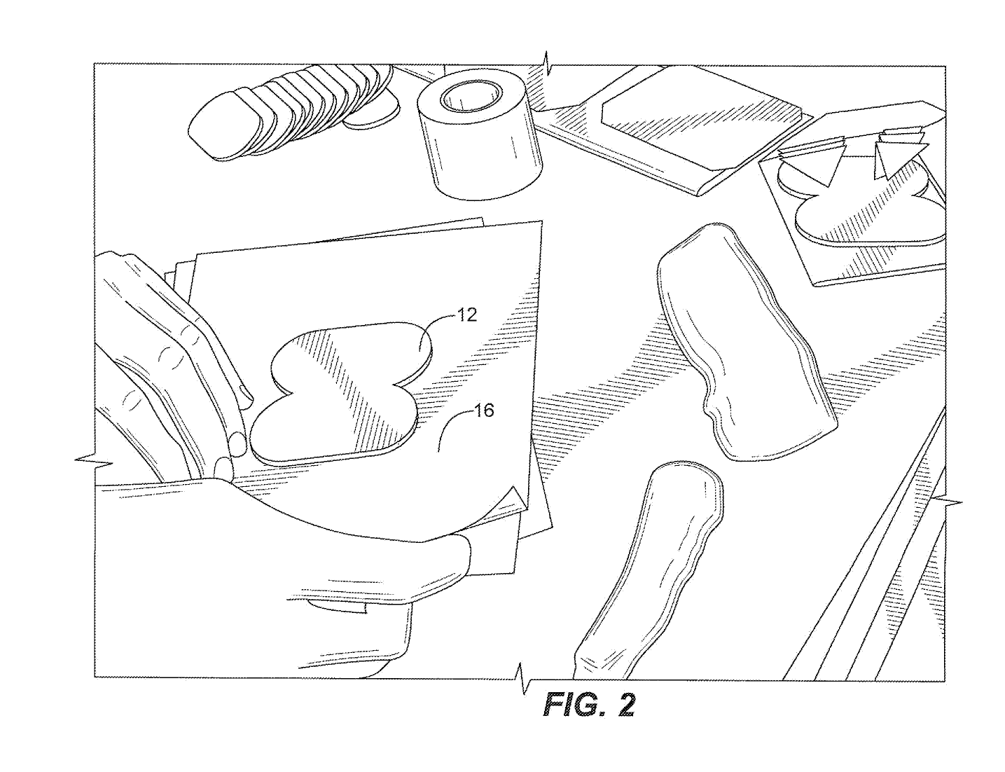

The kit is used as illustrated in the Figures. First, the formable material 14 is placed on a sheet 16 and then wrapped with the sheet 16 (FIG. 2, step S100 FIG. 1A)). The sheet 16 with the material 14 is then placed into bag 12 so that the sheet 16 is in intimate contact with the heating composition 12A (step S102). The sheet 16 is a release sheet made of a thin film of plastic or a thin paper coated with a suitable substance that prevents the sheet 16 from sticking to the material 14 even in the presence of heat. The sheet 16 prevents direct contact between the heating composition 12A and the material 14. The heating composition 12A may be in the form of several components in a powdery or granulated form. A predetermined amount of water (e.g., a spoonful) is then added to the heating composition 12A in bag 12 (Step S104) starting a chemical reaction between the heating composition 12A and the water.

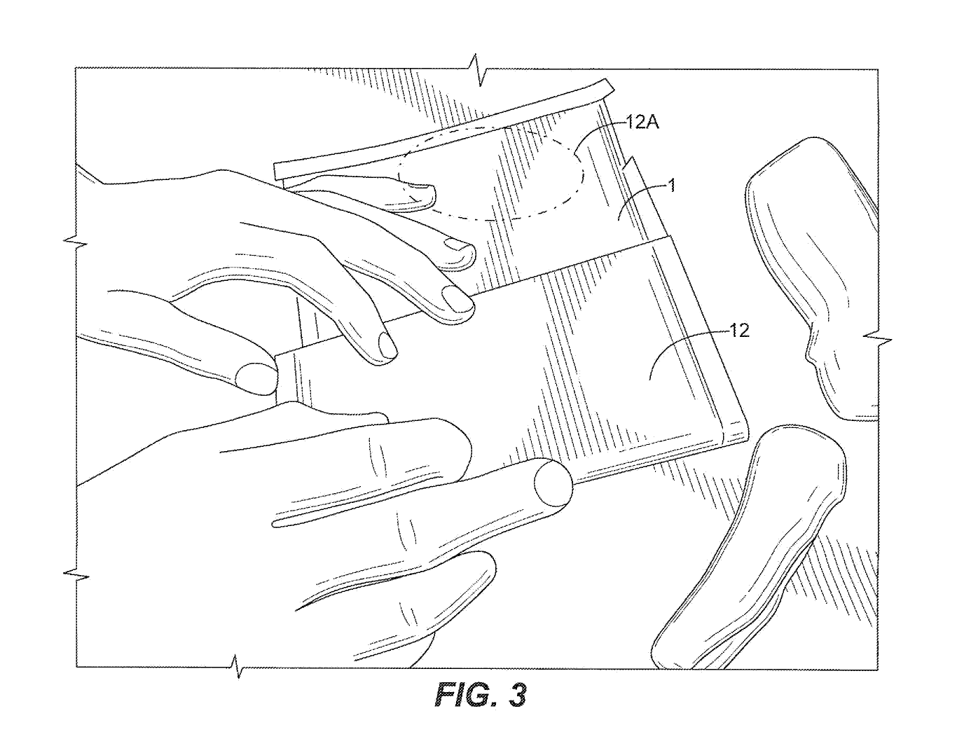

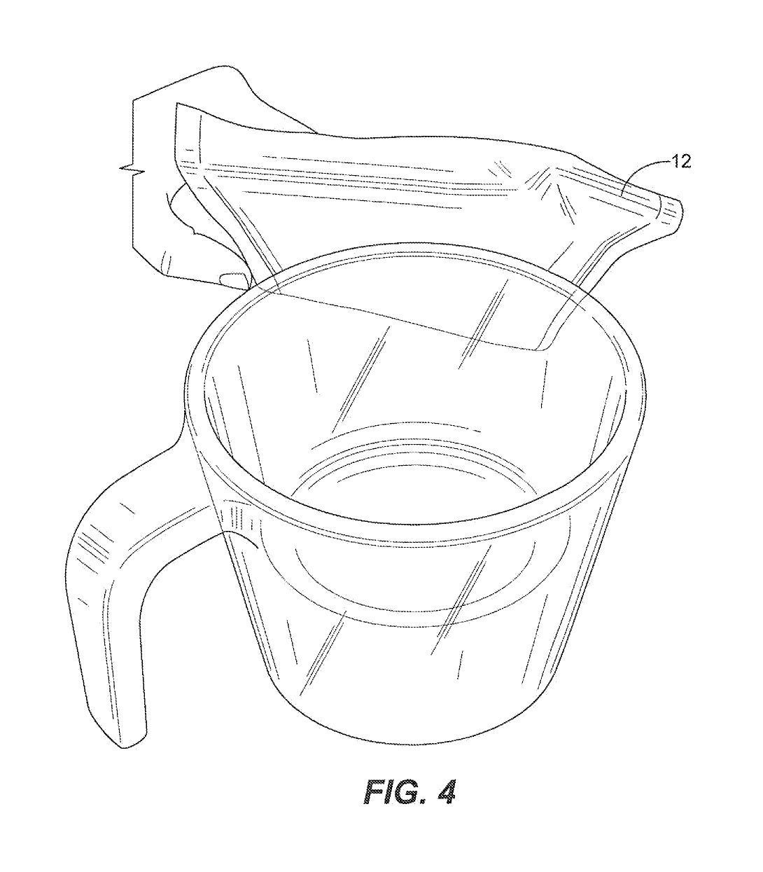

The bag 12 is then closed (FIG. 3), in a conventional manner sufficiently so that the heat generated by the heating composition heats the formable material. The closed bag is maintained in this condition for several minutes to allow the formable material and heat up and soften. (The inventor has found that typically two minutes is sufficient). Preferably during this waiting stage the bag 12 is maintained in an upright position by leaning it against a cup of water 20 (FIG. 4). (Water from cup 20 has been previously poured into the bag as described). During this time, the heating composition 12A reacts with the water and generates heat that heats up the material 14 to about 45-75° C. (steps S106, S108 in FIG. 1A).

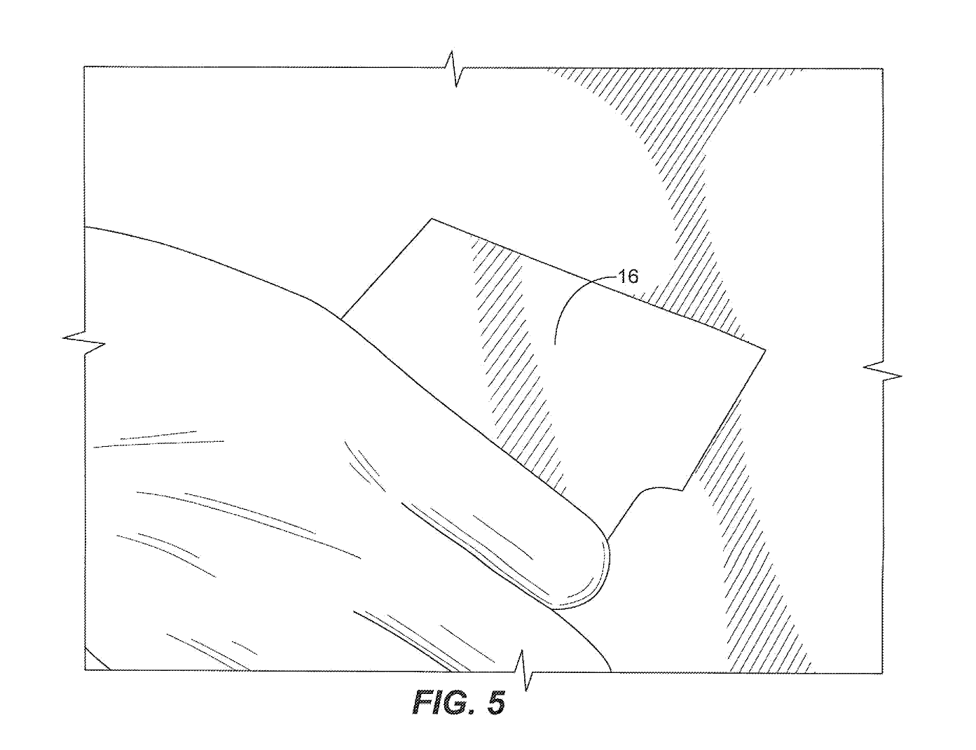

As the material 14 heats up, it becomes very malleable so that it is easy to work it by hand and shape it into any desired shape. After the formable material is heated up, the sheet 16 with the soft formable material is removed from bag 12 (FIG. 5) and the formable material 14 is separated from the release sheet 16. Advantageously, at room temperature, the formable material has a solid opaque look. However when it is in its malleable form, the formable material becomes somewhat translucent. Thus, the person making the mending device can easily determine whether the formable material 14 has been heated up sufficiently.

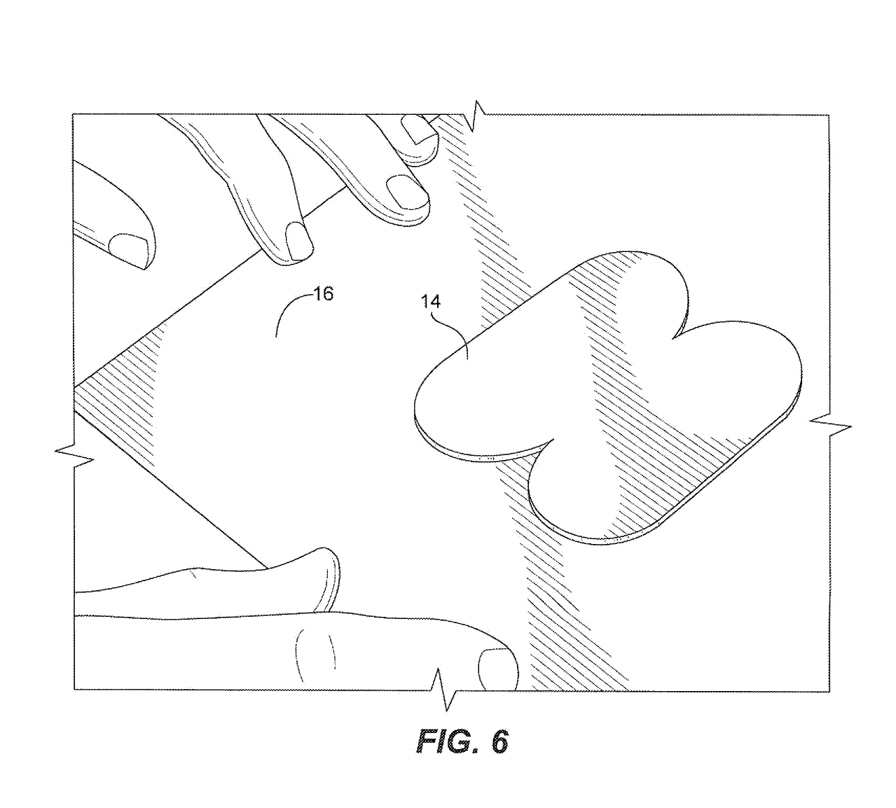

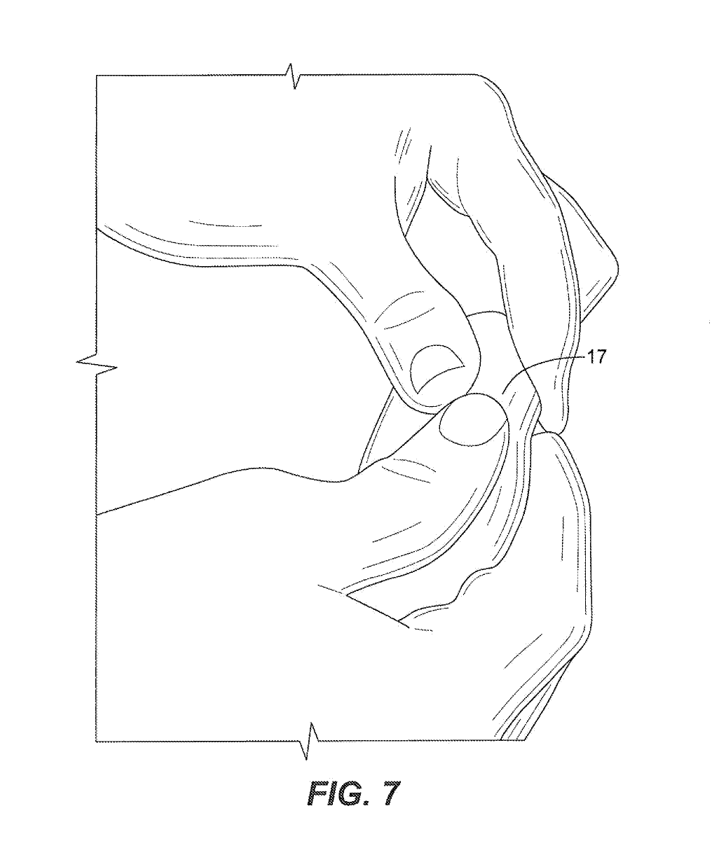

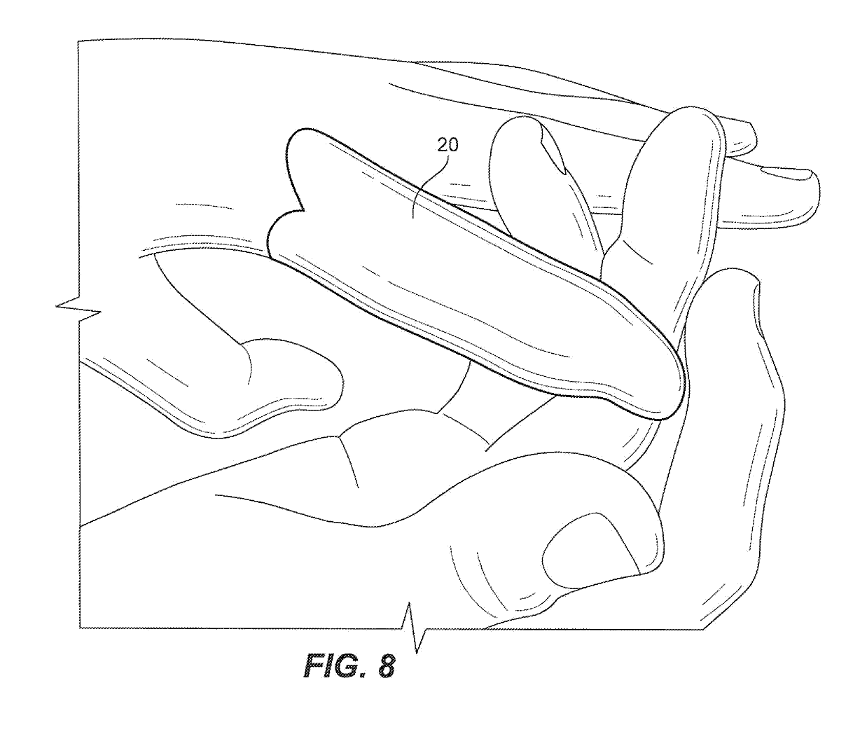

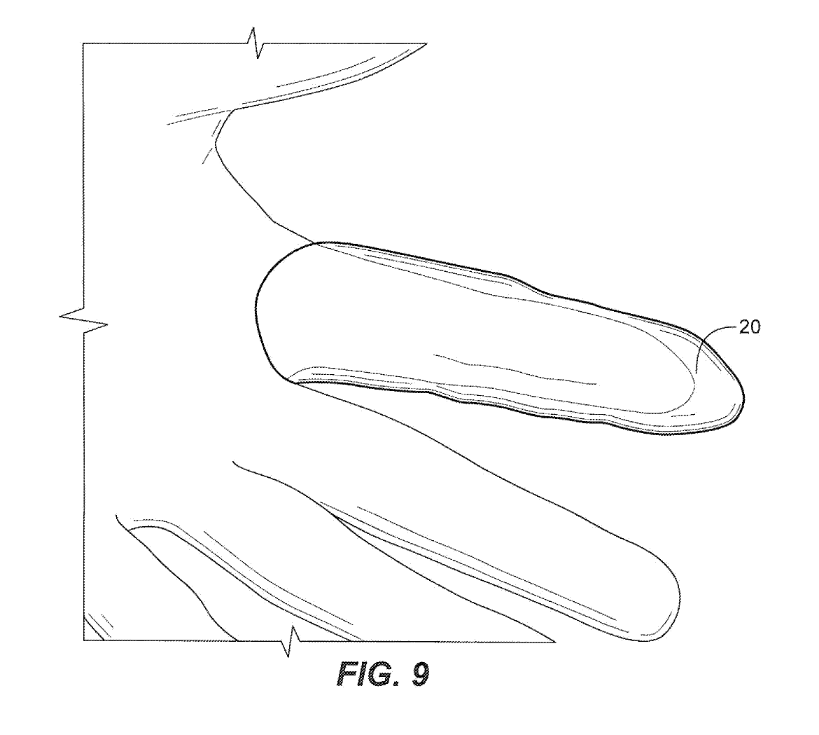

The soft formable material is then shaped manually (FIG. 6) and then molded over a limb, such as finger F (FIG. 7) to form a mending device such as a splint 20 (FIGS. 8, and 9, step S112). Within a couple of minutes the splint 20 cools to room temperature and is set so that is hard and rigid and protects the limb. Importantly, as the splint 20 cools, its solid color returns and is no longer translucent.

In step 114 a determination is made as to whether the splint 20 is sufficient (e.g., large enough to cover a desired portion of the limb, such as a joint) or whether splint needs to be larger. If the splint 20 is large enough then the process is done. Optionally, the limb (e.g., finger F) and splint 20 are wrapped with wrapper 18 in a conventional manner.

Certain heatable and formable materials, such as a material made of polycaprolactone can be formed and re-formed as well as added to an original formation with no apparent or significant loss in physical properties. So, if for some reason the splint 20 is not acceptable (for example, because it is too small), it is removed from the finger F, placed in an appropriate vessel (not shown, step S120) and the molded material is reheated using a microwave or any other means available (step 122). The softened material is then placed on the limb (finger F) and remolded as necessary (step S124).

As shown in FIG. 9, if the splint comes up too small so that it does not cover a sufficient portion of the finger, then more formable material can be added to the splint. In step 114 is more material is required, another piece of the formable material is softened as described above, heated in step S126 and is added to the splint 20 (step 128). This process is made possible by the fact that the one piece of the softened material can be added readily to another piece that has been softened and formed into a splint previously.

The kit and method have been described above for making a splint for the finger of a person. However, the same method can be used to make other mending devices to cover and immobilize other portions of a person's body or to cover the portions the body of an animal.

Finally, while the invention has been described in conjunction with a kit and method for making a mending device for patients, similar kits could be made for mending elongated inanimate objects as well, such as broken electrical connectors.

In the description provided above, the paper 16 is used to insure that the formable material 14 does not get contaminated with the heating composition. Other means may be used to achieve similar results. For example, the heating composition within the bag can be arranged so that it is separated from the formable material by an inner lining or by placing the heating composition in its own pouch, the lining or pouch being made of a water permeable sheet.

This kit could include easily understood instructions for making a mending device such as temporary splint or for repairing or strengthening human or animal bones, tissues, etc.

Numerous modifications may be made to the invention without departing from its scope as defined in the appended claims.

We claim:

1. A kit for making a mending device for mending a body portion of a person or animal, said kit comprising:
a formable material sized for forming said device, said formable material being hard and rigid at room temperature and being malleable when its temperature has been raised to an elevated temperature above room temperature so that it can be formed into said device, said formable material returning to being hard and rigid as it cools down to room temperature; and
a heating source selectively heating said material from room temperature to said elevated temperature.

2. The kit of claim 1 wherein the material has a softening temperature of 45-75° C., and is sufficiently shape-stable at approximately 37° C. to retain a desired shape.

3. The kit of claim 1 wherein the formable material is made of one of a polycaprolactone and a polyvinyl acetate compound.

4. The kit of claim 2, wherein the formable material is in the form of a sheet about 3.5 mm thick.

5. The kit of claim 1 wherein said heating source includes a chemical composition that selectively activated to heat up, said composition being arranged to heat said formable material.

6. The kit of claim 5 further including a bag holding said chemical composition.

7. The kit of claim 5 wherein said chemical composition undergoes a chemical reaction when activated to generate heat for heating said formable material.

8. The kit of claim 5 wherein said composition reacts with water to generate heat.

9. The kit of claim 5 further comprising a separator for separating said formable material from said chemical composition while the formable material is heated.

10. The kit of claim 9 wherein said separator is a sheet of paper.

11. The kit of claim 1 wherein said heating source is a flameless heater.

12. A method for making a mending device for supporting or immobilizing a portion of the body of a human or animal comprising the steps of:
a) providing a formable material having a rigid configuration at room temperature and being malleable at an elevated temperature;
b) heating the formable material to said elevated temperature;
c) molding said formable material at said elevated temperature into a shape required for said mending device; and
d) allowing said shaped formable material to cool to room temperature, wherein at said room temperature the shaped formable material becomes rigid again.

13. The method of claim 12 wherein said step of heating includes inserting saud formable material into a bag, said bag including a heating source, and activating said heating source to heat said formable material.

14. The method of claim 13 wherein said bag includes a chemical composition.

15. The method of claim 14 further comprising activating said chemical composition.

16. The method of claim 15 wherein said step of activating includes adding water to said chemical composition.

17. The method of claim 11 further comprising wrapping said formable material into a protective sheet prior to said heating.

18. The method of claim 11 wherein said formable material is heated from about 37° C. to about 45-75° C.

19. The method of claim 11 wherein said formable material is molded around the desired body portion.

20. The method of claim 11 further comprising heating another piece of formable material; and shaping said another piece of formable material.
